(19) United States
(12) Patent Application Publication
(10) Pub. No.: US 2007/0184086 A1
(43) Pub. Date: Aug. 9, 2007

(54) Biomedical device having crosslinked biopolymer micro pattern and preparation thereof

(75) Inventors: Lung-Jieh Yang, Taipei County (TW); Yu-Cheng Ou, Taipei County (TW)

Correspondence Address: BACON & THOMAS, PLLC, 625 SLATERS LANE, FOURTH FLOOR, ALEXANDRIA, VA, 22314, US

(73) Assignee: Tamkang University, Taipei County (TW)

(21) Appl. No.: 11/449,820

(22) Filed: Jun. 9, 2006

(30) Foreign Application Priority Data
Feb. 6, 2006 (TW) 95103959

(51) Int. Cl. A61K 35/12; A61F 2/02

(52) U.S. Cl. 424/423; 424/93.7

(57) ABSTRACT

This invention proposes a novel technique for fabricating a gelatin micro pattern for cell culture. The gelatin micro pattern is formed by photolithography and then crosslinked with a crosslinking agent such as glutaraldehyde. The gelatin micro pattern can be used as an excellent cell culture platform for in-vitro observations of a certain cluster of living cells or even a single living cell.

### FIELD OF THE INVENTION

The present invention is related to a biopolymer micro pattern for cell culture, and more particularly, it is related to a biopolymer micro pattern used in the biomedical research for the cultivation of a certain cluster of living cells or a single living cell.

### BACKGROUND OF THE INVENTION

In the field of biomedical and genetic research of cell culture, it is critical to make the cells selectively attach to specific location on the substrate. In response to this demand, the technique of forming protein micro pattern on the surface of the substrate has been developed. In this technique, protein is used to allow the cells to selectively attach to the substrate, which in turn generates cell micro pattern, thereby dictating cells to grow at specific location. Therefore, this technique can facilitate the research and observation that are relevant to cell biology.

The currently known methods that are employed to produce protein micro pattern include micro-contact-printing technique, as well as self-assembled monolayer on a micro patterned metal surface. However, these techniques have disadvantages like poor spatial resolution, complicated production procedures, and the resulted protein micro pattern cannot be preserved for a long period of time. Moreover, when the techniques are applied to substrates of large area, the cost can become forbiddingly expensive.

### SUMMARY OF THE INVENTION

A primary objective of the present invention is to provide a technique for forming a biopolymer micro pattern without the drawbacks of the prior art.

Another objective of the present invention is to provide a technique for preparing a biopolymer micro pattern that has high resolution, long preservation period, and high bio-compatibility.

In order to accomplish the above-mentioned objectives A biomedical device having a crosslinked biopolymer micro pattern constructed according to the present invetn comprises a substrate and a crosslinked biopolymer micro pattern attached on the substrate.

Preferably, the biopolymer is gelatin, collagen, or a mixture that contains gelatin or collagen. More preferably, the biopolymer is gelatin.

Preferably, the micro pattern has a resolution between 10 to 1000 μm, and more preferably, between 10 to 150 μm.

Preferably, the substrate is glass or silicone.

Preferably, the crosslinked biopolymer is formed by crosslinking a biopolymer with a crosslinking agent selected from the group consisting of genipin, reuterin, glutaraldehyde, formaldehyde, dialdehyde starch, carbodiimide, and epoxy compound. More preferably, the crosslinking agent is genipin or glutaraldehyde. In one of the preferred embodiments of the invention, glutaraldehyde was used as the crosslinking agent.

Preferably, the device of the present invention further comprises cells grown on the crosslinked biopolymer micro pattern.

The biopolymer micro pattern of the present invention can be widely applied to the field of biomedical research; particularly for the culturing of a certain cluster of living cells or a single living cell. Moreover, it can also help in reducing the inoculation amount for expensive cells and achieving a desired cell density for inoculation, thus giving the present invention wide industrial application.

### BRIEF DESCRIPTION OF THE DRAWINGS

FIG. 1a shows the SEM photograph of the gelatin micro pattern produced by using 45 wt % glutaraldehyde aqueous solution and crosslinking time of 1.5 minutes, according to the method described in Examples of the present invention.

FIG. 1b illustrates the SEM photograph of the gelatin micro pattern produced by using 45 wt % glutaraldehyde aqueous solution and crosslinking time of 1.5 minutes, according to the methods described in Examples of the present invention.

FIG. 2 shows the relationship between crosslinking time and overcrosslinked distance derived from different concentrations of glutaraldehyde aqueous solutions, according to the methods described in Examples of the present invention.

FIGS. 3a to 3d show the SEM photographs of the gelatin micro patterns in accordance with the methods described in Examples of the present invention. FIG. 3a shows the result obtained from using 45 wt % glutaraldehyde aqueous solution and crosslinking time of 1 minute. FIG. 3b shows the result obtained from using 45 wt % glutaraldehyde aqueous solution and crosslinking time of 1 minute. FIG. 3c shows the result obtained from using 45 wt % aqueous glutaraldehyde solution and crosslinking time of 1 minute. FIG. 3d shows the result obtained from using 45 wt % aqueous glutaraldehyde solution and crosslinking time of 1 minute.

FIG. 4a to 4b show the SEM photographs that display the outcome of using two gelatin testing film without micro pattern for cell culture for three days, wherein the gelatin of the testing film in FIG. 4a has been treated with crosslinking agent directly; while the testing film in FIG. 4b has undergone photolithography (applying photoresist→total exposure→developing) before being treated with crosslinking agent.

FIGS. 5a to 5b illustrate the results of utilizing the crosslinked gelatin micro pattern prepared from Examples of the present invention for cell selective growth, wherein FIG. 5a is the SEM photograph taken on the second day of cell culture, and FIG. 5b is the SEM photograph taken on the third day of cell culture.

### DETAILED DESCRIPTION OF THE INVENTION

The present invention discloses a method for preparing biomedical device having acrosslinked biopolymer micro pattern, including the following steps:

a) coating a substrate with a layer of biopolymer;

b) coating a layer of photoresist on the biopolymer layer;

c) imagewise exposing the photoresist layer;

d) developing the exposed photoresist resulting from step c) to form a patterned photoresist layer, so that a portion of the biopolymer layer is exposed;

e) contacting the exposed portion of the biopolymer layer with an aqueous solution containing a crosslinking agent, so that the exposed biopolymer is crosslinked;

f) removing the patterned photoresist layer from the biopolymer layer; and

g) immersing the resulting intermediate from step f) in water or an aqueous solution to remove another portion of biopolymer layer that has not been crosslinked, so that the substrate is formed with a crosslinked biopolymer micro pattern thereon.

An appropriate biopolymer for use in the present invention can be any biopolymers that contain amino groups, in which the amino groups serve as the crosslinking site for the crosslinking agent. Preferable examples include (but not limited to) gelatin, collagen, or a mixture thereof. In one of the preferred embodiments of the present invention gelatin was used as the biopolymer. Humans have been utilizing gelatin for all kinds of purposes for more than 6,000 years, such as using it to make jelly and gummy candy in the food industry, capsules in the pharmaceutical industry, films in the negative used in photography, and facial mask in cosmetic products. Gelatin is obtained and refined from the collagen contained in animal connective tissues, such as the skin of cows and pigs, as well as cartilage or tendon, which means gelatin is a protein that belongs to the collagen family. Although the discovery of gelatin had taken place early on, it still remains a completely novel material with regard to surface micromachining (Lung-Jieh Yang et al., Sensors and Actuators A: Physical, 103(1-2): 284-290, 2003). By treating gelatin with the photolithography technique, which is used frequently in the making of traditional micro devices, followed by a crosslinking treatment with a crosslinking agent, gelatin is imparted with excellent capability in terms of biomedical compatibility, mechanical property, anti-water transmission, and anti-swelling.

In the present invention, the photoresist and its coating method employed in step b), the imagewise exposing employed in step c), the developing employed in step d), as well as the removal of patterned photoresist layer in step f) can be any known methods used in the photolithography technique. The preferable methods are the ones that have the minimal adverse effects on the biopolymer.

The crosslinking agent used in step e) in the present invention can be a natural crosslinking agent or a chemical one that is capable of crosslinking biopolymers contain amino groups. The concentration of crosslinking agent in the aqueous solution and the reaction time varies slightly for different types of crosslinking agents. The main principle for making these variations is decided by whether they can provide sufficient level of crosslinking, so that the crosslinked biopolymer would not be washed off the substrate by the water or aqueous solution used in step g).

Once modified by crosslinking agents, the surface characteristics of organic tissues or proteins would also be altered, thus their structural stability would change as well. The crosslinking agents that are most commonly used for this purpose are formaldehyde, glutaraldehyde, dialdehyde starch, carbodiimide, and epoxy compound.

Genipin can be engendered by using β-glucosidase to remove glucose molecules from geniposide, wherein geniposide is extracted from gardinia fruit. The gardinia fruit is often used in traditional Chinese medicine to treat all types of immune disorders and liver diseases. Some research literature has demonstrated that genipin is an excellent natural crosslinking agent for protein (for reference, see Fujikawa et al., Biotechnology Letter 9: 697-702, 1987). The gardinia fruit has been successfully applied in traditional Chinese medicine and genipin and its related derivatives have been used as colorants in food; hence the toxicity of Genipin should be relatively low. The previous research papers have proved that the cytotoxicity of genipin is much lower than that of glutaraldehyde and other chemical crosslinking agents (for reference, see Sung et al., J Biomater. Sci. Polymer Edn, 10: 67-78, 1999; EP1260237A1).

Taiwan patent application number 89124818 (publication number 550065) discloses a method for utilizing 3-hydroxypropinoaldehyde, otherwise known as reuterin, to crosslink and disinfect a biopolymer to prepare biocompatible implants, substitution material or wound dressing.

In step g) of the method of the present invention, the preferable time for immersing the intermediate in water or aqueous solution is 5 to 10 minutes, wherein the temperature of the water or aqueous solution can be raised in order to accelerate the removal of the portion of biopolymer that has not been crosslinked. For example, the intermediate can be immersed in water with a temperature range between 35° C. and 90° C. for 1 to 3 minutes.

The present invention can be more fully comprehended by reading the detailed description of Examples listed below. It should be noted that Examples only serves the purpose of elucidating the present invention, and are not to be used to limit the scope of the present invention.

### EXAMPLES

In these examples a method for preparing a crosslinked gelatin micro pattern, as well as its application in cell culture were carried out. The steps in this method are listed as follows:

(1) 10 g of gelatin (Sigma Corporation of America, Model G2500 type A bloom 300) was dissolved in 90 ml of deionized water, and filtered prior to being used.

(2) After the glass substrate was cleaned by using the piranha solution, which was made by mixing sulphuric acid and hydrogen peroxide, it was then rinsed to clean off the solution by using de-ionized water. This was followed by spin-coating the glass substrate with a thin film of gelatin solution made in step (1) at 40° C., the film was then allowed to dry at room temperature (for approximately 3 to 4 hours); the thickness of the film was approximately 1.5 μm.

(3) A thin layer of positive photoresist solution (AZ Electronic Materials Co., Model AZ-P4620) was coated on top of the layer of dried gelatin. After the layer of the photoresist had dried, it was exposed by using a photomask at the wavelength of 365 nm and the wattage of 5 mW/cm². The exposure dosage was approximately 250 mJ/cm² and the exposure time was about 30 to 60 seconds. An alkaline solution (KOH-based, AZ Electronic Materials Co., Code: AZ-400K) was utilized for the developing process to define a desired photoresist micro pattern.

(4) The substrate that had been defined with the photoresist micro pattern was immersed in glutaraldehyde solution to carry out time-controlled crosslinking reaction.

(5) Acetone was employed to dissolve and remove the photoresist layer, then followed by rinsing with a lot of deionized water to wash off the unreacted residual of crosslinking agent.

(6) The substrate was then immersed in heated deionized water (approximately 80° C.) to dissolve the portion of gelatin film that has not been crosslinked.

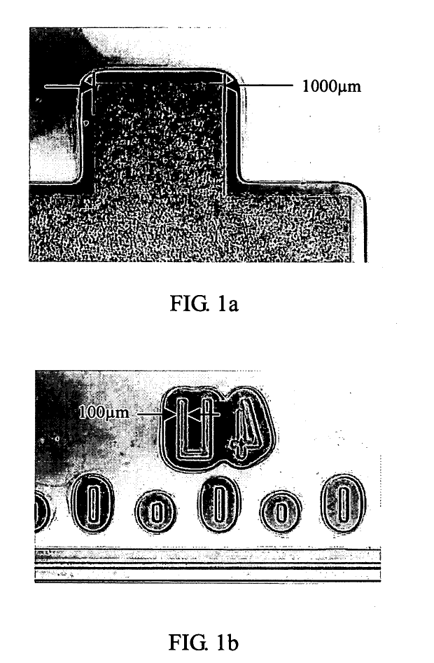

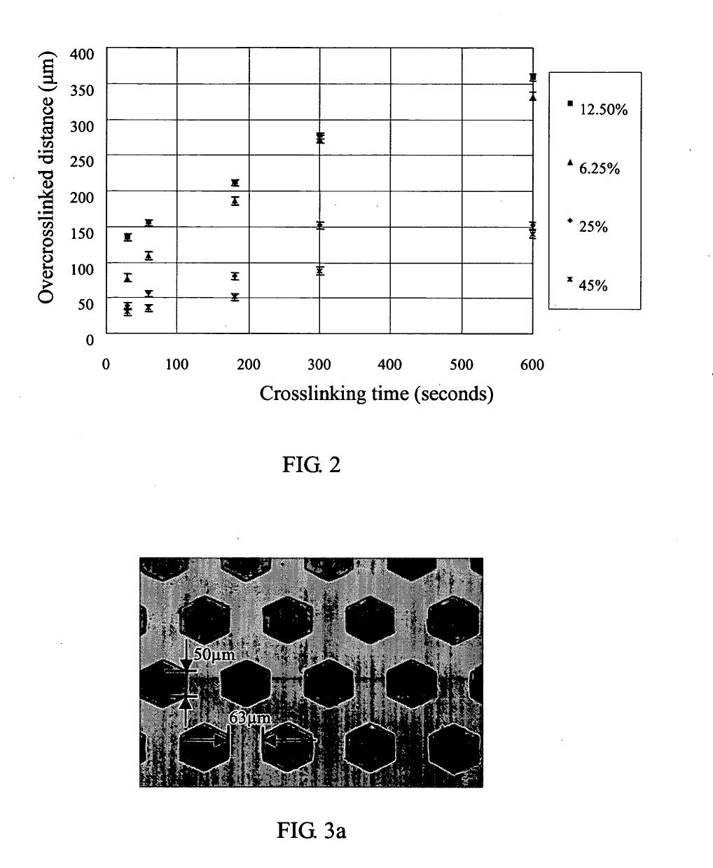

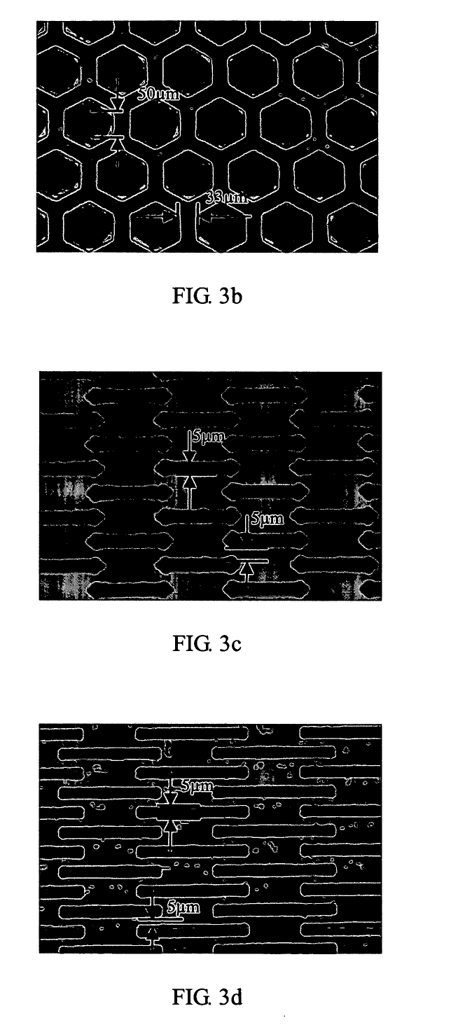

If the crosslinking time was not appropriately controlled, the resulted gelatin micro pattern would end up with excessive overcrosslinks, as shown in FIGS. 1a and 1b, which illustrate the SEM photograph of the gelatin micro pattern produced by using 45 wt % glutaraldehyde aqueous solution and crosslinking time of 1.5 minutes. FIG. 2 shows the phenomenon of overcrosslinked distance under different concentrations of glutaraldehyde aqueos solution and crosslinking time. As shown in FIGS. 3a to 3d, by increasing the concentration of crosslinking agent (for gelatin film with a thickness smaller than 1 μm, the appropriate concentration range of glutaraldehyde aqueous solution is within 25 to 50 wt %) and reducing crosslinking time (for gelatin film with a thickness smaller than 1 μm, the appropriate range of crosslinking time is 5 to 15 seconds), gelatin micro pattern with more precise scale could be obtained. FIG. 3a indicates that glutaraldehyde aqueous solution of 45 wt % and crosslinking time of 1 minute was employed. FIG. 3b shows that glutaraldehyde aqueous solution of 45 wt % and crosslinking time of 1 minute was used. FIG. 3c illustrate that glutaraldehyde solution of 45 wt % and crosslinking time of 1 minute was utilized. FIG. 3d also indicates that glutaraldehyde solution of 45 wt % and crosslinking time of 1 minute was employed.

When the crosslinked gelatin micro pattern prepared in Examples encountered water vapor, its thickness did not show any sign of swelling.

### Result of Tests for Cell Culture:

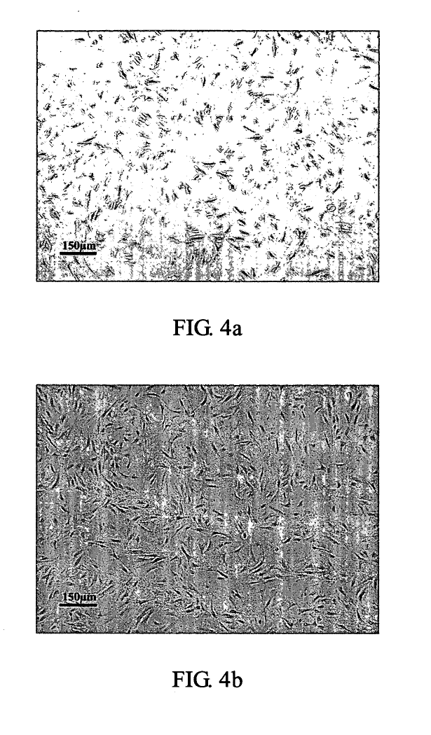

Because organic substances were used in photolithography during the production of gelatin micro pattern, it was necessary to test whether the residual organic substances that remained in the gelatin micro pattern have any negative effects on cell growth. Therefore, two gelatin testing films without micro pattern were used to compare cell culture. One of the testing films had undergone crosslinking reaction directly; while the other one had been treated with photolithography (applying photoresist→total exposure→developing) prior to crosslinking reaction. After the treatments were completed, the two testing films were used to culture mesenchymal stem cell for three days, and then the cell culture results were compared, which are illustrated in FIGS. 4a and 4b. The cell density derived from the testing film that had undergone photolithography was 1.5×10⁴ cell/cm²; whereas the cell density derived from the testing film that had only been treated with crosslinking agent was 1.8×10⁴ cell/cm². In other words, photolithography only makes cell growth density decline 16.7%. The outcome of reduced cell growth density may be resulted from the hydrophobic photoresist that was coated on top of the gelatin during the production. Consequently, the hydrophilicity of the gelatin was reduced slightly, which in turn led to the decline in cell growth density. However, the extent of the overall decline is not clear.

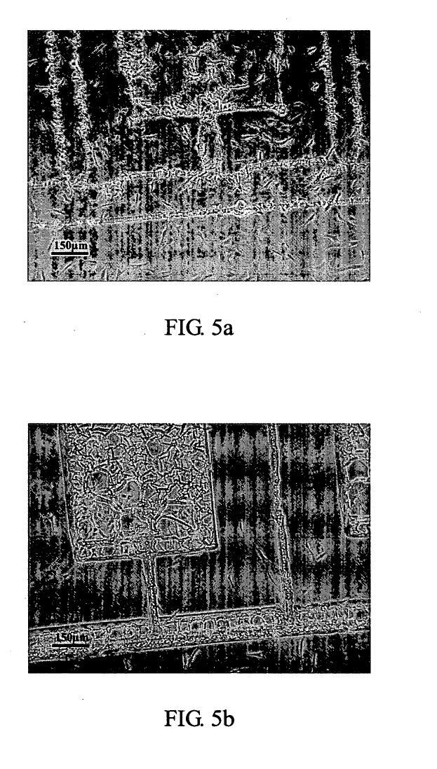

In order to prove the feasibility of utilizing the crosslinked gelatin micro pattern produced by the present invention for cells to grow selectively at specific location, one of the gelatin micro patterns produced in Examples was used to carry out cell culture experiment (in which mesenchymal stem cells were cultured). The cell culture lasted for three days, and the observation of the results were made on the second and the third day of the experiment. As indicated in FIGS. 5a and 5b, the cells still shown even distribution on the second day of cell culture; but later selectively attached and grew at specific locations on the third day of cell culture. The cell density on the surface of gelatin micro pattern was approximately 6.48×10⁴ cell/cm², whereas the cell density on the surface of the glass substrate was merely 400 cell/cm², which means the cell density of the former was a hundred times greater than that of the latter. Therefore, it is clear that substrates with gelatin micro pattern can enhance selective attachment of cells much better than the one without the micro pattern. This result proves that the present invention can indeed control cells to grow at specific location during cell culture.

The present invention has the following characteristics and advantages:

1. Simple Production Process

In comparison with other methods that employ micro-contact-printing technique and self-assembled monolayer on a micro-patterned metal surface to generate protein micro pattern and then micro pattern for cells, the present invention proposes a method for fabricating gelatin micro pattern. This means no additional interfacial agent was required, and the gelatin can be made attach to glass directly, followed by direct fixation on the substrate and then the formation of gelatin micro pattern; the overall production processes are reasonably straightforward.

2. The Cost of Materials is Lower than that of other Biomedical Methods

The method that utilizes crosslinking agents to form the gelatin micro pattern and subsequently generate micro pattern for cells have lower production costs than that of the other biomedical methods, thus its application can be extended to production process that involves larger surface area of substrates and chips.

3. The Inoculation Amount for Expensive Cells can be Reduced

In the present invention, natural materials are used to make the biopolymer, which is subsequently used in combination with the production for micro devices. Therefore, the testing chips can be cut into chips for real use after production, and the size of the chip with gelatin micro pattern can be minimized in order to lower the required inoculation amount for expensive cells.

4. The Preservation Limitation of the Testing Chips and Materials

Generally, in the methods that employ uncrosslinked protein to produce micro pattern for cells, the protein materials and the completed testing chips can both be negatively affected by the environmental temperature, and the testing chips also have limited effective period, which means it cannot be left unused for too long. But in the present invention, the gelatin micro pattern is formed by crosslinking agent directly before cell culture experiment is carried out, thus it can be preserved for a relatively longer period of time. As a result, the gelatin micro pattern of the present invention can greatly facilitate the preparation work of cell culture.

5. High Bio-Compatibility

The biopolymers, such as gelatin, are polymers made of natural materials like animal skins; they are made up by 18 types of amino acids and are long chains composed of approximately 1000 amino acids. The biopolymers have been applied as capsule materials and post-surgical anti-adhesive sheet because of its excellent biomedical compatibility and biodegradability, and its application in cell culture have enormous potential.

6. Low Production Temperature

The temperature of the production process employed in the present invention does not exceed 80° C., which means the present invention can be used in combination with materials and production of micro-processing at lower temperature. Because the production process does not damage the existing micro structure on the biomedical chip; when used in combination with other production processes, it has better flexibility than the other methods.

1. A biomedical device having a crosslinked biopolymer micro pattern comprising a substrate and a crosslinked biopolymer micro pattern attached on the substrate.

2. The device of claim 1, wherein the biopolymer is gelatin, collagen, or a mixture that contains gelatin or collagen.

3. The device of claim 2, wherein the biopolymer is gelatin.

4. The device of claim 1, wherein the micro pattern has a resolution between 10 to 1000 μm.

5. The device of claim 4, wherein the micro pattern has a resolution between 10 to 150 μm.

6. The device of claim 1, wherein the substrate is glass or silicone.

7. The device of claim 1, wherein the crosslinked biopolymer is formed by crosslinking a biopolymer with a crosslinking agent selected from the group consisting of genipin, reuterin, glutaraldehyde, formaldehyde, dialdehyde starch, carbodiimide, and epoxy compound.

8. The device of claim 7, wherein the crosslinking agent is genipin or glutaraldehyde.

9. The device of claim 8, wherein the crosslinking agent is glutaraldehyde.

10. The device of claim 1 further comprising cells grown on the crosslinked biopolymer micro pattern.

11. A method for preparing a biomedical device having a crosslinked biopolymer micro pattern, which comprises the following steps:
a) coating a substrate with a layer of biopolymer;
b) coating a layer of photoresist on the biopolymer layer;
c) imagewise exposing the photoresist layer;
d) developing the exposed photoresist resulting from step c) to form a patterned photoresist layer, so that a portion of the biopolymer layer is exposed;
e) contacting the exposed portion of the biopolymer layer with an aqueous solution containing a crosslinking agent, so that the exposed biopolymer is crosslinked;
f) removing the patterned photoresist layer from the biopolymer layer; and
g) immersing the resulting intermediate from step f) in water or an aqueous solution to remove another portion of biopolymer layer that has not been crosslinked, so that the substrate is formed with a crosslinked biopolymer micro pattern thereon.

12. The method of claim 11, wherein the immersing in step g) is carried out for a period of 5 to 10 minutes.

13. The method of claim 11, wherein the immersing in step g) is carried out in water or an aqueous solution of 35 to 90° C. for a period of 1 to 3 minutes.

14. The method of claim 11, wherein the biopolymer in step a) is gelatin, collagen, or a mixture that contains gelatin or collagen.

15. The method of claim 14, wherein the biopolymer is gelatin.

16. The method of claim 11, wherein the crosslinking agent in step e) is selected from the group consisting of genipin, reuterin, glutaraldehyde, formaldehyde, dialdehyde starch, carbodiimide, and epoxy compound.

17. The method of claim 16, wherein the crosslinking agent is genipin or glutaraldehyde.

18. The method of claim 17, wherein the crosslinking agent is glutaraldehyde.

19. The method of claim 18, wherein the aqueous solution containing a crosslinking agent is a glutaraldehyde aqueous solution having a concentration of 25-50 wt %, and the contacting is carried for a period of 5 to 60 seconds.
